(19) United States
(12) Patent Application Publication
(10) Pub. No.: US 2006/0047326 A1
(43) Pub. Date: Mar. 2, 2006

(54) Application of specific neuromodulation waveforms to reduce symptoms of neurological disorders

(75) Inventor: James Walter Wheeler, Vista, CA (US)

Correspondence Address: James W. Wheeler, 1787 Kings Road, Vista, CA, 92084, US

(73) Assignee: James Walter Wheeler

(21) Appl. No.: 10/927,450

(22) Filed: Aug. 27, 2004

(51) Int. Cl. A61N 1/18

(52) U.S. Cl. 607/48

(57) ABSTRACT

A preferred form of the invention can reactivate proprioception system neural pathways by means of specific neuromodulation waveforms, application points and procedures. The improvement and advancement of this invention is the ability to significantly improve the quality of life of people with movement disorders caused by loss of neural transmission in the proprioception system. Unlike present transcutaneous electrical stimulation (TENS) devices this invention addresses movement disorders as a closed loop control system malfunction and not as a pain management problem. This invention has been shown to relieve leg neuropathy, reduce Parkinson's disease tremor and rigidity, and allow frozen shoulders to be completely restored.

### CROSS-REFERENCE TO RELATED APPLICATIONS

| | | | |
|---|---|---|---|
| US Patent; 5,052,391 | Oct. 1, 1991 | Silverstone et al | 607/46; 607/66; |
| | | | 607/76 |
| US Patent; 4324253 | April 1982 | Gregne et al. | 128/421 |

Federally Sponsored Research and Development—No federally sponsored research and development applies.

### BACKGROUND OF THE INVENTION

(1) Field of Invention

This invention relates to transcutaneous electrical stimulation and more particularly to a new approach to using neuro-modulation waveforms and procedures to reactivate myelinated nerve fibers essential to restoring proprioception system functionality.

(2) Description of the Prior Art

Networks of nerves (the nervous system) direct and monitor every body system and cell, governing all movement, sensation, thought, and emotion. These many networks function properly in a healthy body. However, whenever the flow of neuronal impulses is disrupted a change in the body's function occurs. The most drastic examples involve injury to the spinal chord that either puts pressure on, or completely severs the nerves. Other cases involve inflammation of the nerve, or nerves, which may be caused by some kind of general poisoning (lead, arsenic, alcohol) or a virus.

Many neurological impairments are caused by degenerative diseases such as Alzheimer's, Parkinson's, Multiple Sclerosis, etc. These diseases are generally attributed to degeneration of specific cells in the brain. Alzheimer's is attributed to slowly progressive neural atrophy of the cerebral cortex; Parkinson's disease is attributed to slowly progressive degeneration of the substantia nigra and corpus striatum, with a dopamine deficiency; Multiple Sclerosis is attributed to patchy de-myelination of the white matter in the brain and spinal chord, marked by unpredictable exacerbations and remissions. These diseases are currently managed by drugs but are deemed incurable. There seems to be little effort to mitigate these brain-induced symptoms through attempts to restore neurologic function throughout the many related neural-muscular systems.

The flow of neural messages throughout the proprioception system controls all muscle movement. A description of these processes follows. The brain functions are as follows:

The basal ganglia and cerebellum are large collections of nuclei that modify movement on a moment-to-moment basis. Motor cortex sends information to both, and both structures send information right back to the cortex through the thalamus.

The Basal Ganglia is inhibitory (restricts movement) while Cerebellum is excitatory (creates movement). The balance between these two systems allows for smooth, coordinated movement. A disturbance in either system will show up a movement disorder. To sit still, you must put the brakes on all movements except those reflexes that maintain posture. To move, you must apply a brake to some postural reflexes, and release the brake on voluntary movement. In such a complicated system, it is apparent that small disturbances can throw the entire system out of balance, often in unpredictable ways.

The Basal Ganglia is a collection of nuclei deep in the white matter of the cerebral cortex. The Substantia Nigra is included in the Basal Ganglia and produces Dopamine, which is critical for normal movement. The Substantia Nigra degenerates in patients with Parkinson's disease. Three symptoms usually associated with Parkinson's disease are tremor, rigidity, and bradykinesia. The tremor is most apparent at rest. Rigidity is the result of simultaneous contraction of flexors and extensors, which tends to “lock up” the limbs. Bradykinesia (slow movement) is a difficulty initiating voluntary movements, as though the brake cannot be released.

The Cerebellum is the central computer for controlling movement. It compares what you thought you were going to do (according to the motor cortex) to what is actually happening throughout your body according to proprioception (position and motion sensor) feedback, and tries to correct the differences in a smooth manner. The Cerebellum also is partly responsible for motor leaming, such as properly hitting a golf ball.

The Cerebral Cortex (Motor Cortex) sends information to both the Basal Ganglia and the Cerebellum and both structures send information right back to the cortex through the Thalamus. The motor control signals travel down the spinal chord to control the alpha motor neurons, to activate muscles. Sensory signals travel up the spinal chord to report sensor status, such as muscle length and tension.

Inside the spinal column the spinal nerves branch off from the spinal chord in pairs. Each of the nerves has two roots: the one in front is the ventral root and carries only motor impulses, the one in back is the dorsal root and carries sensory signals. Within the spinal nerve there is two-way traffic, since it carries both sensory and motor signals for the part of the body it serves. At any given time there can be a mixture of different sensory messages being passed along. But once the nerve feeds the signals into the spinal chord, they are sorted and separated into groups of nerve fibers. One tract handles motor impulses from the brain. Other tracts carry sensations of pain, temperature, proprioception, etc.

However, to execute orders from the brain and also to ensure that the brain has the proper sensory information, a complex network of neuro-muscular pathways and sensors must be functional. The following summarizes how these functions work:

Assume that a load is added to a limb and stretches the muscle. The muscle spindle fires a series of pulses as the muscle is stretched signaling a change in length. Golgi Tendon Organs (GTO) also fire, signaling increased stress on the tendon. Alpha motor neurons are activated, causing extrafusal fibers of the muscle to contract, shortening the muscle back to its original length. The muscle spindles are no longer stretched, and are therefore inactivated as the muscle shortens. GTOs remain under tension (due to the load and active muscle contraction) and continue to fire, signaling the amount of tension experienced on the tendon. Gamma motor neurons are activated, causing a contraction of the muscle spindles, in proportion to the change in muscle length. This re-sets the spindles, allowing them to become active in case the shortened muscle changes length. More receptors are recruited as the intensity of the stimulus (stretch or tension) increases to ensure that the signals exceed the threshold required to ensure that muscle damage will not occur. If the tension detected by the GTOs exceeds a safe level, GTO inputs override the inputs of the muscle spindles. This inhibits activity of both the alpha motor neurons and the gamma neurons, and leads to a relaxing (lengthening) of the extrafusal fibers and of the muscle spindles. The muscle tension is reduced to a safe level and the brain is then tasked to determine an alternative approach the handling the load.

The motor control fibers (alpha nerves) directly drive the muscle to which it is connected. A motor endplate terminates in a series of synaptic knobs, which are separated from the muscle tissue by the synaptic space. Within each knob are packets (vesicles) containing the neuro-transmitter acetyclcholine. The arrival of a nerve impulse makes the vesicles release acetycholine into the synaptic space. This in turn, brings about the release of sodium ions, which act on the muscle filaments and make them slide over each other in muscular contraction. Meanwhile, an enzyme acetycholinesterase breaks down the acetycholine and makes it inactive. This resets the mechanism in time for the arrival of the next nerve impulse, needed to keep the muscle contracting.

Muscles are present in opposing groups, as illustrated by the biceps and triceps of the upper arm. Contraction of the biceps bends the elbow, while contraction of the triceps straightens it. When a muscle is being intentionally stimulated to contract, it is called an agonist. Opposing muscles are called antagonists. For instance, when you flex your biceps, it is the agonist, while the triceps is the antagonist. Note that any given muscle will be an agonist in one situation, but an antagonist in another. Also note that when an agonist contracts, the antagonist naturally gets stretched. The response to this stretching is what goes wrong in spasticity.

This natural stretching of antagonist muscles is necessary for movement, but too much stretching can damage the muscle. Two related signaling pathways help prevent too much, and too little, stretch of the antagonist. First, while an agonist is contracting, the spinal cord sends inhibitory signals to the antagonist to prevent it from contracting while it is being stretched out. Second, the antagonist sends sensory signals back to the spinal cord to indicate how much it is being stretched. When the antagonist is stretched too far or too fast, these sensory signals override the inhibitory signals, and set off protective contractions in the antagonist to prevent muscle damage. These two antagonist-regulating pathways help to insure that the intended movement can occur, but cannot damage surrounding muscles.

Spasticity occurs when these two antagonist-regulating pathways are improperly controlled, usually due to upper motor neuron damage. First, the pathway that normally inhibits antagonist contraction becomes less active than normal, leading to unwanted co-contraction of antagonists during movement of an agonist. This prevents smooth movement and full range of motion in the agonist. Second, the stretch reflex becomes hyperactive, so that the antagonist is likely to contract even when stretched only slightly. As a result of loss of inhibition and hyperactive stretch reflexes, movement becomes difficult to control, and muscles may remain stiff and contracted for long periods of time.

Each nerve fiber has a cell body containing a nucleus that sprouts root like dendrites. An axon extends from a “hillock” on the cell body, and is wrapped with myelin sheath (a chain of Schwann cells separated by gaps at the nodes of Ranvier). Terminal buttons at the end of the axon link to other nerve cells.

Most nerve cells have a single axon and a large number of dendrites. The dendrites are approximately 1.3 mm (0.05 in.) long and terminate in thousands of smaller dendrite spines. Electrical impulses arrive from other nerve cells through these dendrites. All the electrical messages (series of impulses) are integrated in the cell body. The cell body creates a new series of impulses containing the appropriate information and passes it along the nerve.

Most of the nerve fibers that carry signals in the body (except in the brain) are myelinated. Nerve impulses travel along a myelinated nerve fiber chain by a process called “saltatory” conduction, not through the synaptic process used to connect nerve fibers together. The essential aspect of this whole process is the rapid conversion of chemical potential energy to electrical potential energy.

These myelinated fibers are like a chain of sausages. The links (Schwann cells) are approximately 2 millimeters long with small (1.0 micrometers long) interruptions (nodes of Ranvier) between the Schwann cells. These nodes of Ranvier are the sole places where ion transfer takes place. Typical values of propagation times quoted for myelinated axons are 6 to 9 m/s per micron diameter. A 10 micrometer diameter myelinated fiber will propagate an action potential impulse at between 60 and 90 meters/second along the nerve chain as long as there is not an interruption of the process. Myelinated nerve diameters are typically between 5 and 20 microns.

The propagation of a nerve impulse along an axon begins when the synapse receives neurotransmitters from nerve endings nearby. The neuron then increases its internal potential, setting off a chain of events which is repeated for each node of Ranvier as the nerve impulse “jumps” down the axon (this is known as “saltatory” conduction).
1. Voltage gated sodium (Na) channels open when the membrane potential raises about 20 mV above the rest potential; this potential is called the “threshold”. Na ions rush in for about 1 ms; positive feedback (the membrane potential continues to rise above the threshold) keeps the channels open until the cell interior becomes positively charged to approximately 30 mV before the Na channels close. About 2000 ions enter per channel during the Na influx. Note that until the membrane potential drops below the threshold, the neuron cannot react to further stimulus.
2. The Na ions migrate a small distance away from the node, and additional Na ions move toward the node within the interstitial fluid. The increased positive ion concentration inside the node increases the membrane potential at both neighboring nodes (with very minute effects further down the axon on both sides). The “downstream” node reaches threshold and the process continues there. The “upstream” node reaches threshold as well, but its threshold has been raised high enough by its firing that it does not fire again. In this way, the nerve impulse propagates down the axon, maintaining its intensity until it causes the release of neurotransmitters at the nerve endings.
3. The Na channels close when the voltage peaks, and potassium (K) channels open and let K into the interstitial fluid. They remain open for about 1.5 ms, until the membrane potential overshoots its initial value slightly. During this time, the neuron's “firing” threshold is much higher than normal. Finally, Na pumps restore the concentrations that existed before firing.

This saltatory conduction process will be prematurely terminated when there is a malfunctioning node of Ranvier. This can be caused by an injury that either causes physical damage or a traumatic event that causes the node to lose its charge. Considerable research has been conducted to (1) develop mathematical models of neuron conduction for both myelinated and non-myelinated nerves and (2) to empirically validate these models. This research provides a solid foundation to define most of the waveform parameters required to effectively reactivate inactive nerve channels and restore physical functionality by electrical stimulation. Little research has been done to reactivate defective nerves.

To date transcutaneous electrical stimulation devices use very simple waveforms that stimulate sensor nerves using waveform parameters that provide pain relief and in some cases stimulate neurotransmitter production. They however have one very significant failure; they do not penetrate the myelinated nerve sheath, but only stimulate the synapse and dendrites.

Western medicine defines the nervous system as a system of interrelated systems. Some are physically separate, others differ only in function. The brain and spinal chord make up the Central Nervous System (CNS). The rest of the system is called the Peripheral Nervous System (PNS). It is impossible to obtain a broad picture of how this complex and sophisticated nervous network controls our every thought, feeling and movement. However, Asian medicine attempts to do this by making an analogy to repetitive and circulatory functions such as blood flow and breathing. Asian medicine relates overall function of the stomach, gall bladder, intestines, and etc., to acupressure points and energy (Qi) channels. Additionally Asian medicine seems to account for turbulent flow within these channels.

Historical evidence reveals that the ancient Chinese physicians knew that acupuncture was physiologically based, affecting blood and vital air (breath) circulation as well as nerve function. In the 1980's, investigators related the effects of acupuncture to the stimulation of at least 10 neural structures. Evidence also shows that complete denervation obliterates the effects of acupuncture. To date there seems to be no one-to-one correlation but there are many physical points where nerves and acupressure paths are essentially the same. Neuroanatomic acupuncture correlates traditionally described points and channels with neurologic pathways as understood today. Acupressure points are known to also be points where the electrical conductivity is highest (lowest resistance) and are believed to coincide with a node of Ranvier.

### SUMMARY OF THE INVENTION

A preferred form of the invention can reactivate proprioception system neural pathways by means of specific neuromodulation waveforms, application points and procedures. The significant improvement and advancement of this invention is the ability to significantly improve the quality of life of people with movement disorders caused by loss of neural transmission in the proprioception system. Unlike present transcutaneous electrical stimulation (TENS) devices this invention addresses movement disorders as a closed loop control system malfunction and not as a pain management problem. This invention has been shown to relieve leg neuropathy, reduce Parkinson's disease tremor and rigidity, and allow frozen shoulders to be completely restored.

In accordance with one of the aspects of this invention, the present invention pertains to a method of Neuromodulation stimulation in which a electrical signal's frequency is variable over the frequency range that couples effectively into the nodes of Ranvier over a range of myelinated nerve sheath diameters. The frequency range shall by sufficiently high (approximately 25 kHz) to couple into a single node of Ranvier and yet sufficiently low to couple into nodes of Ranvier separated by up to 2 meters (approximately 250 Hz). Additionally the amplitude of the carrier signal shall be adjustable at each frequency to match the node of Ranvier threshold level at each frequency.

In accordance with one of the aspects of this invention, the present invention defines an amplitude-modulated waveform that effectively causes sufficient Na ion transfer at the nodes of Ranvier to cause the node's threshold to be sufficiently elevated to cause production of an action potential. This waveform is expected to be different for a healthy resting cell than for a malfunctioning cell. The primary variables are pulse width and pulse repetition rate.

In accordance with one of the aspects of this invention, the present invention defines a series of weighted pulses that simulate a series of action potentials. The shape of the action potential is known to be approximately 1.5 milliseconds long and to have specific rise and fall times. The envelope of this action potential will be filled with individual pulses of variable widths as required to support the above production of action potentials.

In accordance with one of the aspects of this invention, the present invention defines a series of simulated action potentials to create neural messages representing authentic sensor and motor control messages. Preliminary research indicates that message lengths vary from as few as 1 or 2 sporadic action potentials to as many as 200 specifically timed action potentials. The actual encoding and decoding of neural messages is an ongoing research area that can be accommodated in the range of waveform parameters chosen.

In accordance with one of the aspects of this invention, the present invention defines a methodology for choosing the specific Neuromodulator unit electrode placement. Essentially one electrode is to be placed on one end of the neural pathway and another is to be placed progressively along the neural pathway in a manner that allows one to determine the ability of the chosen neural pathway to transmit the electrically generated waveform. Generally the neural pathways underlay the meridians described in Asian medicine. However, this is not a limiting application.

### BRIEF DESCRIPTION OF THE DRAWINGS

FIG. 1 is a generalized description of a simplified myelinated nerve, emphasizing nerve structural elements.

FIG. 2 is a generalized description of a neuron's action potential, emphasizing key voltages and timing relationships.

FIG. 3 is a description of a representative weighted pulse sequence that is central to this invention.

FIG. 4 shows use of the Neuromodulator with advanced waveforms and electrode placement for median nerve diagnosis and reactivation.

FIG. 5 shows use of the Neuromodulator with advanced waveforms and electrode placement Asian medicine stomach channel diagnosis and reactivation.

FIG. 6 is a block diagram of the preferred embodiment of the Neuromodulator capable of implementing the waveforms and procedures of this invention.

### DETAILED DESCRIPTION OF THE INVENTION

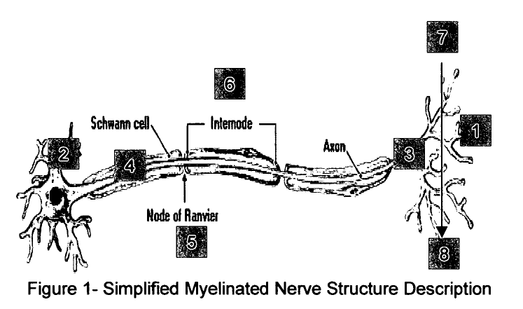

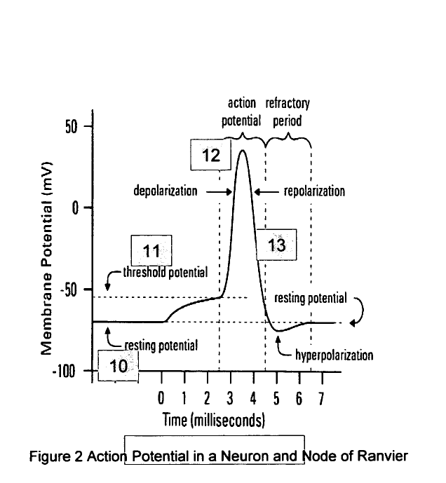

Referring to FIG. 1, a dendrite complex 1 connects to a nerve cell 2 (neuron) through an axon 3. The dendrites can be electrically stimulated by placing electrodes in locations 7 and 8 and applying a current of a few milliamps. The electrical current will travel between the pairs of electrodes 7 and 8 and when sufficiently high will excite the dendrite such that an action potential (as shown in FIG. 2) is created. The action potential will then travel down the nerve toward the cell body. When all of the Schwann cells 4 and Nodes of Ranvier 5 along the nerve are healthy, the action potential will hop from one Node of Ranvier to the next at a propagation velocity of approximately 30 to 180 meters per second and up to distances of several meters. However, when the saltatory conduction process breaks down, the action potential will travel along the axon (by electron conduction) at a much slower rate and for only a few inches until the action potential dissipates. Consequently, damage along a myelinated nerve sheath that extends to as little as an inch can disable the entire nerve.

One of the aspects of this invention uses the above conduction principles to reactivate myelinated nerves. This is achieved in part by applying an electrical current at specific frequencies and of sufficient intensity to raise the external voltage at the Nodes of Ranvier such that the nodes will generate an action potential. The specific frequency that is most effective will be that which has a half wavelength that extends across the length of the damaged section of the nerve. This damaged area may extend only the length of one Schwann cell (approximately 2 millimeters) or may extend along a patch of scar tissue that may be several inches long. An action potential traveling at 100 meters per second will cross the internodal space 6 in approximately 0.02 milliseconds. The corresponding frequency that will provide maximum coupling is 25 kHz. A frequency slightly above or below this frequency will not provide maximum coupling and will not be as effective. Consequently it is important to be able to vary the frequency and determine that which provides the most effective coupling. A frequency of 250 Hz has a half wavelength of 0.2 meters (approximately 7.8 inches) and is low enough to extend across to length of most areas of nerve damage. Action potential propagation velocity is a function of the myelinated nerve's diameter and varies from as low as 30 meters per second to as much as 180 meters per second. Consequently, the optimum range of frequencies is much larger than 250 Hz to 25 kHz. However, since the effective coupling impedance is frequency dependent the full range of frequencies is not required.

It is believed that one of the aspects of this invention is the ability to reactivate a neuron and/or node of Ranvier that can't create an action potential. FIG. 2 shows the essential characteristics of an action potential in a neuron. A healthy neuron at rest has a voltage potential across its membrane of approximately −70 mV 10. When something happens to decrease that negative voltage to approximately −50 mV the threshold potential 11 is reached and the neuron will “fire”. As described earlier, when the threshold potential is reached sodium (Na) ions rush into the neuron for approximately 1.0 ms and the action potential voltage 12 goes positive to approximately 30 mV. At the voltage peak, potassium (K) channels 13 open for approximately 1.5 ms and the cell returns back to the resting potential. This electrochemical reaction occurs throughout the nervous system.

It is believed that two conditions can cause a neuron to be inactive. The first being the cell's inability to support a potential across the membrane. This would essentially act as an electrical short circuit and is possibly representative of scar tissue. The second is the inability of the cell's Na or K channels to open and close. This may be due to lack of calcium and other nutrients. However, case studies have shown that normal neural conduction can be restored by electrically stimulating inactive neurons. Often the neurons continue to function normally after neural stimulation.

Another aspect of the invention is the use of a range of pulse widths and amplitudes to cause electrical stimulation of both active and inactive neurons. The ability to stimulate an inactive node of Ranvier depends upon its state of health. The energy required to depolarize a health neuron is believed to be significantly less than that of an inactive neuron.

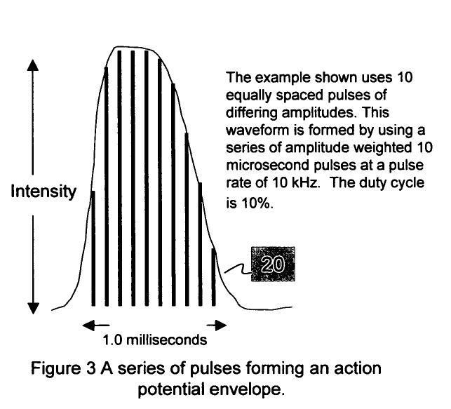

The preferred embodiment of this invention creates pulses that have a rise time of approximately 1.0 microseconds and pulse widths variable from 10 microseconds to 1.0 millisecond. Pulses having these characteristics will allow for a diverse range of waveforms that provide many treatment options. In general transcutaneous electrical stimulation devices only require pulse lengths on the order of 10 to 100 microseconds at current intensities of a few milliamperes to depolarize a healthy neuron. However, pulse lengths of up to 1.0 milliseconds and current intensities of up to 50 milliamperes are often required to reactivate a damaged neuron. Another aspect of this invention is that a series of pulses will be used to fill the envelope of a simulated action potential. FIG. 3 is an example of this waveform. As discussed earlier, the frequency (pulse repetition rate) should be adjusted to stimulate the nodes of Ranvier. The pulse width is then adjusted to establish the desired duty cycle. A maximum duty cycle of 50% is envisioned. Note that a 1.0 millisecond pulse will fill the envelope.

Another aspect of the invention is the formation of a simulated neural message by using a series of the simulated action potentials previously described. Both the frequency and timing (spacing) of the action potentials determine the nature of the message transmitted. Research indicates that proprioception sensors send an average of 11 spikes (action potentials) per second. These spikes are often, but not always grouped into packets approximately 0.1 seconds long. The preferred embodiment of this invention allows for complete freedom in designing the spike frequency and intervals, including random patterns. The intent is to not merely reactivate myelinated nerves but to also reestablish communications within the entire proprioception system.

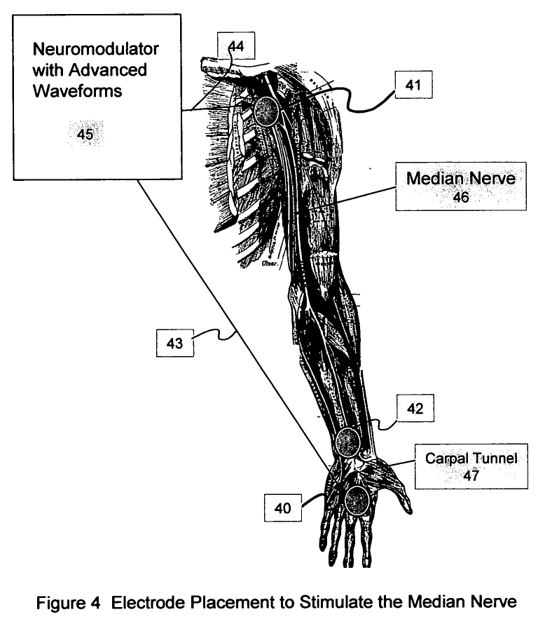

Another important aspect of this invention is the methodology for choosing the placement of the transcutaneous electric stimulation electrodes. The preferred embodiment uses a Neuromodulator (a special TENS unit) that can produce the range of waveform parameters described above at sufficient intensity to reactivate inactive nerve cells. FIG. 4 shows an representative application to stimulate the median nerve. One electrode 40 is placed on the palm of the hand over a series of nerves that connect to the median nerve. Another electrode 41 is attached on the shoulder near the other end of the median nerve. Note that the median nerve goes through the carpal tunnel that is known to put pressure on the median nerve and can cause numbness of the fingers.

Electrodes 40 and 41 are connected to the Neuromodulator unit 45 by electric cords 43 and 44. In this example the intent is to determine if the median nerve is functional. A healthy median nerve will conduct electrical current between electrodes 40 and 41 at current intensity levels as low as a few milliamperes when the example waveform shown in FIG. 3 is used. When there is a neural blockage, like that possible by carpal tunnel compression, there will initially be no or minimal conduction. In the preferred embodiment of this invention, conduction will be evidenced by both an indication on a current meter contained in the Neuromodulator unit and by a tingling sensation at one or both electrodes. The recommended diagnosis procedure is to connect the Neuromodulator using electrodes at locations 41 and 42 and then determine if there is conduction along the portion of the median nerve that does not pass through the carpel tunnel. If conduction is achieved, the nerve damage is localized to the carpal tunnel area. One should then verify this by connecting the Neuromodulator unit to electrodes 40 and 42 and determining if there is conduction. Often what seems to be a carpel tunnel condition is caused by a lack of conduction farther up the median nerve.

Once the location of the blockage is determined, the preferred procedure is to increase the Neuromodulator's unit's waveform duty cycle to 50% and to vary the pulse repetition frequency slowly over the entire frequency range to determine the frequency or frequencies that provide maximum conduction. This procedure is continued at increased Neuromodulator output voltage levels until current flow begins. Often current flow can be reactivated in as few as several minutes. Once current flow is achieved, the procedure should be repeated beginning at a reduced voltage level and reduced duty cycle.

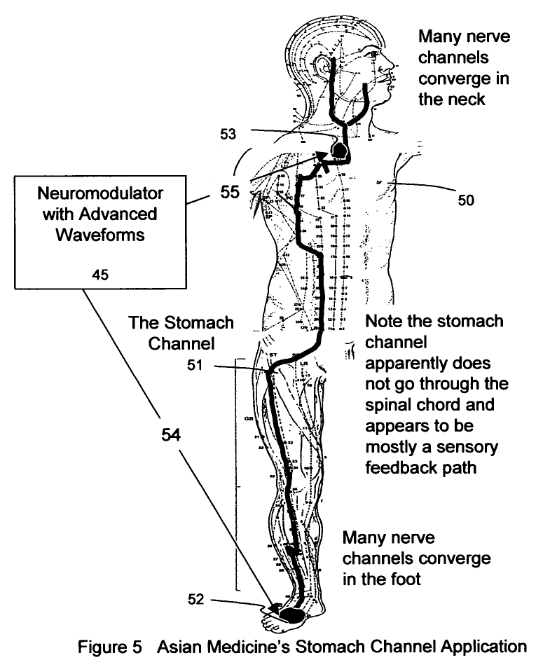

The above is only an example but is typical of the process. Another way to determine electrode placement is by use of Asian medicine charts of the meridians. Attaching the Neuromodulation unit's electrodes at different locations along the meridians is similar to that for the median nerve described above. FIG. 5 shows a representation of the Asian Medicine Stomach Channel 51. When electrodes 52 and 53 are connected to the Neuromodulator 45 by cables 54 and 55, it is possible to conduct electrical current over the length of the stomach channel. This channel appears to be of special importance in the proprioception system and blockage may cause movement disorders. Injuries to the feet, stomach, and neck can disrupt neural communications from the feet to the head along the stomach channel. One can locate the blockage (if any) using the procedures described for the meridian nerve application. Often communications can be restored by use of the Neuromodulation waveforms.

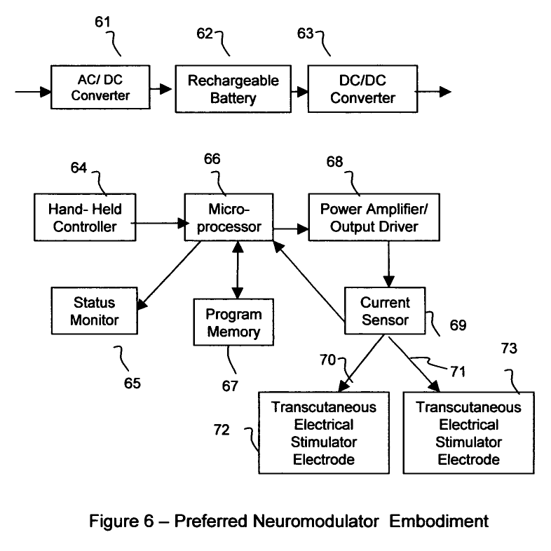

FIG. 6 is a block diagram of the preferred embodiment of the Neuromodulator unit 45 shown in FIGS. 4 and 5. A brief description follows.

The preferred embodiment uses an AC/DC converter 61 to convert normal 115 Vac electricity to approximately 12 Vdc by means of a commercially available isolated device. The 12 Vdc will be used to charge battery 62 such that the Neuromodulator can operate for several hours while disconnected from the AC source. DC/DC converter 63 regulates the battery voltage and provides the voltages required to power all of the Neuromodulator's electronics.

The hand-held controller 64 provides the primary user interface. Functions such as: On/OFF, mode selection, amplitude, pulse width, pulse rate etc. will be selected using digital controls as with a remote TV controller. The remote hand-held controller will transmit and receive messages from the Neuromodulator's microprocessor 66. The microprocessor 66 is a programmable high-speed processor capable of producing the waveforms required to produce effective neural stimulation. The microprocessor's control program and intermediate data will be stored in the program memory 67. The microprocessor will produce pulsed waveforms and associated amplitude control signals. The microprocessor will also receive data from the current sensor 69 and adjust signal strength to implement the modes and signal levels required to implement the hand-held controller 64 selections. A status monitor 65 will display status information as produced by the microprocessor. As a minimum status will include: battery power condition, mode selection, current levels, and possibly propagation velocity.

The Power Amplifier/Output Driver 68 will transform the microprocessor's digital signals to the appropriate analog signals required to efficiently drive the transcutaneous electrical stimulator electrodes 72 and 73. The Power Amplifier is expected to produce bipolar pulses with a minimal DC component. The output pulses should be at least 100 V peak to peak with rise-times less than 0.5 microseconds. The Output Driver 68 will drive the transcutaneous electrical stimulator electrodes at peak currents of up to 80 milliamperes. The transcutaneous electrical stimulator electrodes 72 and 73 are envisioned to be commercially available silver-silver chloride high impedance electrodes designed for high frequency applications.

Presently preferred embodiments of the invention have been described above with a degree of specificity. It should be understood, however, that this description has been made by way of preferred example and that the invention itself is defined by the scope of the appended claims.

What I claim as the invention is:

1) a method for restoring neuro-muscular function by means of applying special frequencies and waveforms designed to penetrate the sheath of myelinated nerves.

2) a method to restore saltatory conduction in myelinated nerve fibers by inducing an electric charge at the nodes of Ranvier.

3) A method for restoring proprioception system functionality by stimulating neuro-muscular channels along the nerve structures including those that underlie the meridians of Asian medicine.

4) A preferred embodiment of the Neuromodulator (a transcutaneous electrical stimulation device) to achieve the above claims.
